(19) United States
(12) Patent Application Publication
(10) Pub. No.: US 2013/0228185 A1
(43) Pub. Date: Sep. 5, 2013

(54) GARMENT WITH BARRIER DEVICE

(71) Applicants: Pauline Ann Yardley Middlehurst, Seattle, WA (US); Gregory Paul Martin, Seattle, WA (US)

(72) Inventors: Pauline Ann Yardley Middlehurst, Seattle, WA (US); Gregory Paul Martin, Seattle, WA (US)

(21) Appl. No.: 13/778,821

(22) Filed: Feb. 27, 2013

Related U.S. Application Data
(60) Provisional application No. 61/605,671, filed on Mar. 1, 2012.

(51) Int. Cl. A61F 5/37

(52) U.S. Cl. CPC A61F 5/37
USPC 128/871

(57) ABSTRACT

A method and apparatus is disclosed herein for a garment with a barrier device. In one embodiment, the garment comprises an upper body garment having a neck opening, two sleeves or arm hole openings, a back, and a compartment located on the back of the upper body garment, where the compartment has a surface therein located in location of the back that encompasses an area that includes at least the shoulder blades of an individual when worn by the individual. The garment includes a barrier device contained within the compartment, where the barrier device has protrusions that contact the surface when the individual initiates movement into a supine sleeping position.

### PRIORITY

The present patent application claims priority to and incorporates by reference the corresponding provisional patent application Ser. No. 61/605,671, titled, “SleepTZ Side Sleeping Shirt and Anti-rollover Device,” filed on Mar. 1, 2012.

### FIELD OF THE INVENTION

Embodiments of the present invention relate to the field of anti-snoring devices; more particularly, embodiments of the present invention relates to a garment with a barrier device to promote sleeping on one's side.

### BACKGROUND OF THE INVENTION

Snoring is a problem for many people. One of the key contributors to snoring is supine (back) sleeping. For many reasons supine sleeping has been cited as a significant problem for those who snore, people who suffer with sleep apnea, people who should not sleep on their backs such as those who are pre and post-operative and even pregnant women. There are many medical studies that explain the benefits of side sleeping.

There is a wide range of solutions including mouth guards, nose strips and nasal vents for keeping the nasal passages open, chin straps to keep the jaw in place to discourage snoring, including all manner of pillows to be used for the head, neck, knees and full body pillows, devices known (as “Positional Sleeper” and “Positional Sleeping Aids”), medical devices and surgical procedures for those who are unfortunate enough to suffer with Obstructive Sleep Apnea (“OSA”). More specifically, positional sleeping aids are highly recommended by medical professionals to aid people who tend to snore more when they sleep in the supine position. Side sleeping is a recommended remedy for snoring and some cases of OSA.

Even with the use of pillows and other sleep aids available on the market, supine sleeping is still a problem for many. The conscious decision to side sleep is not available to the sleeping individual.

Products in the market include the Zzoma® OSA Positional Sleeper, the Rematee Neoprene Bumper Belt with Shoulder Straps and the Rematee Shirt with the bumper pockets. There are limitations with these currently available products. For example, the Zzoma® positional sleep aid is large and creates a barrier to intimacy, significantly affecting and modifying the sleep habits of both partners. Furthermore, the position of the barrier that discourages the subject from assuming a supine position is not optimally placed. The Zzoma® and Rematee positional belts and the Rematee Shirt place the obstruction low enough on the torso to allow the sleeping person to achieve supine sleeping by either rolling inside the device or rolling onto the barrier.

Belt style positional devices have the problem of allowing the subject to move inside the belt which enables supine sleeping. With the belt style positional sleep aids of the prior art, often times to be effective, the belt needs to be secured tightly around the torso to help minimize the movement of the wearer within the confines of the belt. There is a possibility of the belt being secured so tightly as to restrict breathing. This can compromise the effectiveness, trading side sleeping for restricted breathing.

Another sleep issue present with existing designs is the problem of sleeping in neoprene or other non-breathable fabrics, causing perspiration underneath the belt which can make the belt uncomfortable and allow the subject to turn within the belt to achieve supine sleeping. The Rematee Bumper Belt and the Rematee Shirt both use air filled balls to attempt to keep the subject side-sleeping. The air filled balls allow the subject to rollover and assume a supine position, providing a cushion effect that is comfortable to the sleeping person once the supine position is attained.

Lastly, the position of the rollover barriers in existing products is not optimally placed. The positional belts and the existing shirt design place the barrier low on the torso to allowing the wearer to move achieve supine position by either rolling inside the device or rolling onto the barrier. At least one positional sleep aid is so large that it creates a tent effect within the bed coverings causing a cold draft down the back side of the wearer and, potentially, for their sleeping partner. At least one positional sleep aids has sharp corners that can dig into or be uncomfortable to the partner sleeping next to the wearer.

### SUMMARY OF THE INVENTION

A method and apparatus is disclosed herein for a garment with a barrier device. In one embodiment, the garment comprises an upper body garment having a neck opening, two sleeves or arm hole openings, a back, and a compartment located on the back of the upper body garment, where the compartment has a surface therein located in location of the back that encompasses an area that includes at least the shoulder blades of an individual when worn by the individual. The garment includes a barrier device contained within the compartment, where the barrier device has protrusions that contact the surface when the individual initiates movement into a supine sleeping position.

### BRIEF DESCRIPTION OF THE DRAWINGS

The present invention will be understood more fully from the detailed description given below and from the accompanying drawings of various embodiments of the invention, which, however, should not be taken to limit the invention to the specific embodiments, but are for explanation and understanding only.

FIG. 1 illustrates one embodiment of a barrier device for a garment.

FIG. 2 illustrates one embodiment of a garment that houses a barrier device.

FIG. 3 illustrates the garment of FIG. 2 where the grey shaded area outlines the printed area on the back of the garment as previously described in FIG. 2.

FIG. 4 illustrates one embodiment of a garment of FIGS. 2 and 3.

### DETAILED DESCRIPTION OF THE PRESENT INVENTION

Embodiments of the present invention includes a garment that encourages side sleeping in a comfortable, non-invasive manner. Embodiments of the invention encourage side sleeping by using a physical barrier that discourages the sleeping individual from assuming a supine position. This is an advantage for people who have been told by doctors to side sleep as opposed to supine sleeping.

Embodiments of the invention include a garment that has been modified to include a barrier device. Embodiments of the invention have a low profile that allows for partner sleeping and cuddling while wearing the device. Embodiments of the invention provide a safe, gentle stimulus to the sleeping person to deter them from rolling onto their back, thereby maintaining a more optimal side-sleeping position.

In the following description, numerous details are set forth to provide a more thorough explanation of the present invention. It will be apparent, however, to one skilled in the art, that the present invention may be practiced without these specific details. In other instances, well-known structures and devices are shown in block diagram form, rather than in detail, in order to avoid obscuring the present invention.

### Garment Embodiments and Features

In one embodiment, a garment that covers the upper body is modified so that the back of the garment between the shoulder blades is imprinted with an ink or gel solution that provides two significant properties to the design of the shirt. First, the imprint provides a sticky surface for the barrier device (described below) to sit on. Second, the imprint reinforces the material of the shirt to provide a non-stretchy, stable surface for the barrier device to sit on, thereby creating the trampoline effect described below.

The non-stretchy, stable surface for the garment (e.g., shirt) is an advantageous design feature because this creates tension when the wearer attempts supine sleeping. The feet or other protrusions of the barrier device are inert until the wearer begins the motion to move to a supine sleeping position. The movement to a supine sleeping position causes the feet (or other protrusions) of the barrier device to grip the printed surface of the garment creating tension over the printed area. In one embodiment, the only points of contact of the barrier device to the back of the garment are the feet (protrusions) on the printed area of the garment against the wearer's back. The effect of the tension that is applied to the printed area causes a trampoline effect, the effect of which is that the wearer “feels” like they are next to a solid surface and prompting a movement away from supine sleeping without disrupting sleep or causing pain.

In one embodiment, the garment does not have an imprint, and instead the material on or attached to the garment need only be rigid enough to create the trampoline effect. In another embodiment, this can be accomplished by using a different fabric insert or, in combination with the printed area, by use of a stable fabric that by itself can create the necessary tension.

In one embodiment, the garment is made of a stable, woven polyester that does not stretch or shrink for the back of the garment, with the fabric at the back of the garment being compensated for its lack of stretch in the back fabric by making the front of the garment out of a stretchy fabric to accommodate an individual's body contours. The front of the garment may be made of any material whether woven, knit, interlock or any other type of fabric made of natural or manmade materials.

Other additions to the garment include a compartment in which the barrier device is inserted and housed. In one embodiment, such a compartment is designed and fitted to snugly hold the barrier device in place against the body of the wearer and allows the barrier device to be optimally placed at the upper back in the area of the shoulder blades.

In one embodiment, to keep the barrier device in place, the lower part of the compartment is fitted with a belt. When worn, the belt may provide a stabilizing effect that ensures the positional efficiency of the barrier device.

### Barrier Device Embodiments

In one embodiment, the barrier device comprises a lightweight rigid rectangle of high density foam that has been contoured to provide a barrier to rolling over while having minimal contact with the wearer. Alternatively, the barrier device may be circular, square, an oval or other shaped article.

This minimal contact provides several significant advantages:
1. Allows for air flow so the wearer does not get hot and perspire under the device.
2. Provides space for natural back and spine movement as the wearer breathes and moves naturally in their sleep. During normal sleep with the shirt fitted correctly, the barrier device does not come into contact with the spine.
3. Creates the tension barrier with the printed area on the garment.
   An Example Embodiment of the Garment with the Barrier Device

In one embodiment, the shape of the barrier device is a brick or in the shape of a rectangular box. However, in one embodiment, the barrier device may be in any shape that restricts the wearer from rolling onto their back and allows for minimal contact with the wearer, but where the contact creates tension on the printed area of the garment, allows for air flow and has a cavity which allows for movement and has no contact with the spine.

In one embodiment, a gel-based printing solution is used to create the tension area. The printing may include regular screen printing ink, specialty inks, or printing or impressions that create a surface for the protrusions of the barrier device to sit on or adhere to. In this case, gels, polyurethane, or any other adhesions whether natural or synthetic applied to the fabric that create a surface for the protrusions of the barrier device to sit on and/or adhere to.

Note the tension area may be created using any methodology such as, for example, screen printing a solution to the area, inserting a more rigid piece of fabric, using the original garment fabric as the tension base by making the entire garment or the back or part of the back of the garment out of such fabric, reinforcing the tension area by any method such as stitching, gluing, heat sealing, and/or applying any other surface to create the tension area.

In one embodiment, the pattern design of the tension area consists of a wide perimeter of gel-based printing shaped like a rectangle. The four corners of the rectangle have the 2″ squares printed to create a platform on which the feet of the barrier device rest. The inside of the rectangle is printed with a fishnet design to give rigidity to the fabric to create the tension area. The design of the printing to create the tension area is not limited to the fishnet design and may be created by any method including circles, waves, squares, triangles, honeycomb, letters, numbers, icons, graphics, or any other geometric or non-geometric design or any combination thereof Also note that in one embodiment, any pattern may be used in which the printed area, different fabric area, or added tension area come into contact with the protrusions of the barrier device.

The closure of the compartment may be secured at any position on the box by any method, including but not limited to Velcro®, buttons, snap fasters, flaps, envelope containment, etc. However, in one embodiment, a zip fastener closure method is used and located at the midpoint on the side of the compartment.

In one embodiment, a T-shirt is used as the garment. However, the design can be attached to any garment that covers the upper body, such as, for example, a shirt, T-shirt, pajama top, nightdress, dress, camisole, bra, dress, jacket, sweater, vest, backpack, or any other garment.

In one embodiment, cotton fabric is used to make the garment. The garment can be made of cotton, bamboo, silk, polyester, nylon, rayon, linen, or any other man made or natural fibers or any combination thereof.

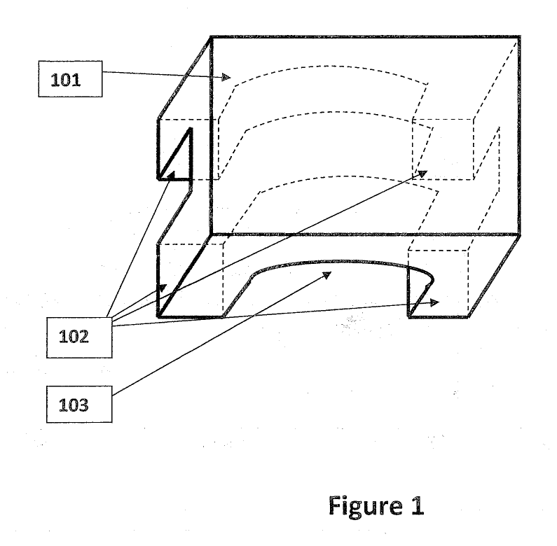

In one embodiment, the garment includes the barrier device depicted in FIG. 1. As shown in this example the top of the dense foam block is indicated by the arrow pointing from box labeled (101). The dimension of this block give the viewer a cross sectional view from the top down where the grey shaded areas, indicated by the arrows from the box labeled (102) define the feet of the cut out block. The arrow from the box labeled (103) indicates the cut out part of the box which provides the channel that creates air flow and a bridge over the spine. In one embodiment, the block is constructed from dense foam which is comfortable against the skin while providing the necessary resistance to prevent the wearer from achieving a supine position.

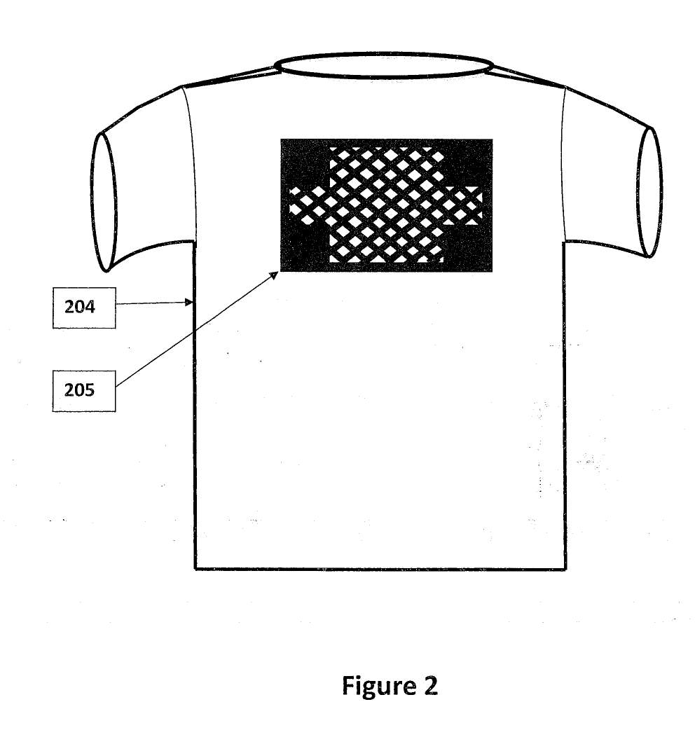

FIG. 2 describes one embodiment of a garment (204) that houses the barrier device as described in FIG. 1. The garment (204) that covers the upper body and item (205) indicates the printed area on the back of the garment which provides the base for the barrier device to sit on thereby, creating the “trampoline effect” when the wearer tries to move to a supine position.

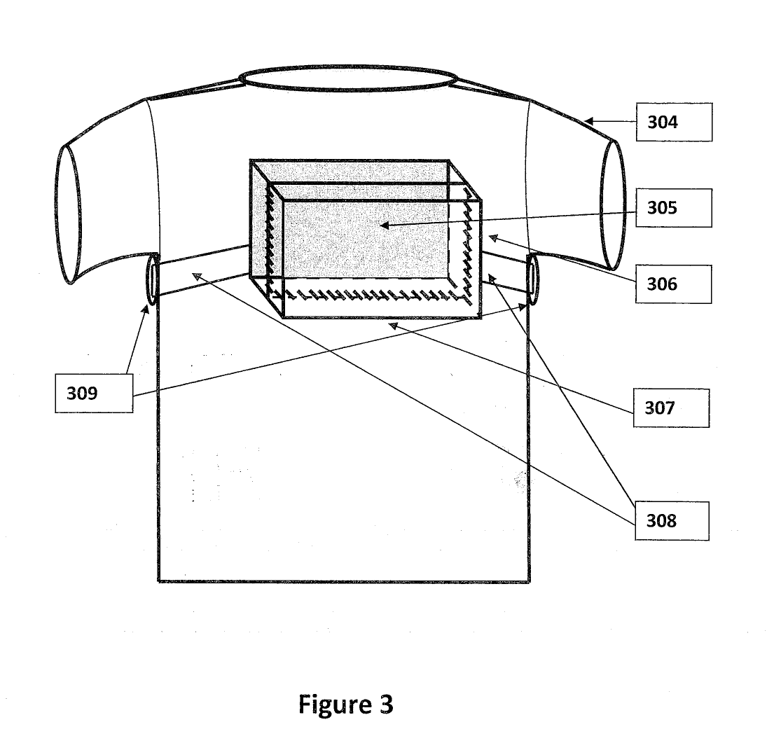

FIG. 3 shows one embodiment of the same garment (304) as described in FIG. 2 where the grey shaded area indicated by (305) outlines the printed area on the back of the garment as previously described in FIG. 2. Item (306) indicates the compartment or box that is attached to the garment (304) by any method and is designed specifically to hold the barrier device as described in FIG. 1. Item (307) indicates the zipper opening that is contained within three sides of the box compartment (306) to allow insertion of the barrier device into the box compartment. Item (308) shows the belt that holds the box (306) containing the barrier device in place on the torso of the wearer. The belt stabilizes the position of the device securely during sleep. Item (309) indicates the belt loops on either side of the garment holding the belt (308) in place.

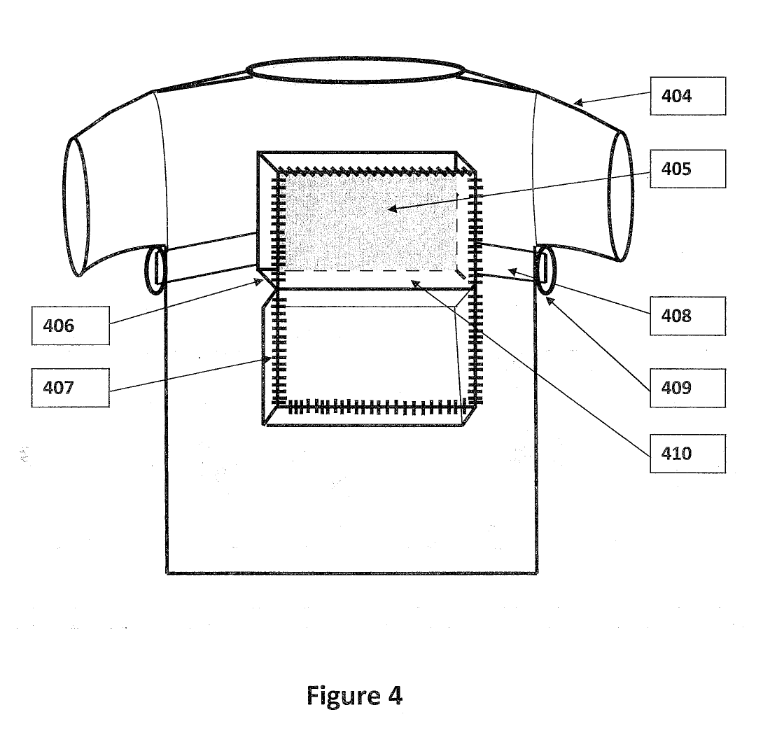

FIG. 4 shows one embodiment of the garment as previously described in FIGS. 2 and 3 where item (404) indicates the garment, item (405) indicates the printed area as shown in grey, item (406) indicates the box compartment to hold the barrier device, item (407) indicates the zippered opening on the compartment opening on three sides, item (408) indicates the belt, item (409) indicates the belt loops and item (410) indicates the area inside the box compartment to house the barrier device as shown in FIG. 1.

Tension is created when the wearer initiates movement to supine sleeping, pressure is placed on one edge of the barrier device which in turn causes the feet to grip the printed area (405) on the back of the garment and at the base of the box compartment (406). The grip caused by the feet of the block against the bottom of the box compartment creates a tension on the fabric against the back of the wearer causing the sensation of being against a large object encouraging the wearer to return to side sleeping. In one embodiment, the barrier device is large enough to prevent the wearer from easily or comfortably, assuming a supine position. The location of the barrier device on the torso allows for comfortable partner sleeping and adequate draping of bed covers to prevent cold drafts.

In one embodiment, a separate pillow sits on top of the barrier device and acts as a cushion barrier between the barrier and the person sleeping next to the wearer. This pillow does not adversely affect the efficacy of the design and is provided for the comfort of the wearer's sleep partner.

Embodiments of the invention encourage side sleeping. Several features of embodiments of the invention include the following:

Embodiments of the invention allow the wearer to enjoy a sleeping garment that is comfortable and made of generally used sleeping fabrics that are comfortable.

Embodiments of the invention allows for air flow between the wearer and the barrier device so the wearer does not get hot and perspire under the device.

Embodiments of the invention are ergonomically designed to provide for natural back and spine movement as the wearer breathes and moves naturally. During normal sleep, with the shirt fitted correctly, the barrier device does not come into contact with the spine.

Embodiments of the design create the “trampoline effect” as described herein to encourage side sleeping and discourage supine sleeping.

Embodiments of the invention do not create the ability for the wearer to move the barrier device out of the way to attain a supine sleeping position.

Embodiments of the invention allow the wearer to be in a comfortable garment that does not need to be cinched tightly to be effective.

Embodiments of the invention use breathable fabric, and because the barrier device has minimal contact with the torso of the wearer, there is sufficient airflow to maintain a comfortable body temperature.

Embodiments of the invention uses dense foam that provides a barrier effect that inhibits supine sleeping effectively, in most cases without disrupting the sleep of the wearer.

Embodiments of the invention have a low profile that does not inhibit the draping of bed covers, thereby eliminating the potential drafts which may be caused by other larger profile devices.

Embodiments of the invention are compact, effective and is positioned high enough on the body of the wearer to allow intimate relations and other affectionate sleeping positions. The social aspects of partner sleeping are not significantly compromised by the garment described herein.

Embodiments of the invention allow the barrier device to be placed high enough on the torso to be effective. When the wearer initiates movement to supine sleeping, the barrier device between the shoulder blades high enough on the torso which discourages the full action of rolling to a supine position.

Embodiments of the design use a belt, strap or other devices to secure the optimal placement of the barrier device such that it remains located in the upper back region and does not migrate up during sleep behind the neck or head, or down past the ribcage.

Whereas many alterations and modifications of the present invention will no doubt become apparent to a person of ordinary skill in the art after having read the foregoing description, it is to be understood that any particular embodiment shown and described by way of illustration is in no way intended to be considered limiting. Therefore, references to details of various embodiments are not intended to limit the scope of the claims which in themselves recite only those features regarded as essential to the invention.

We claim:

1. An upper body garment having a neck opening, two sleeves or arm hole openings, a back, and a compartment located on the back of the upper body garment,
the compartment having a printed surface therein located in location of the back that encompasses an area that includes at least the shoulder blades of an individual when worn by the individual;
a barrier device contained within the compartment, the barrier device having protrusions that contact the printed surface when the individual initiates movement into a supine sleeping position.

2. The upper body garment defined in claim 1 wherein the protrusions grip the printed surface when the individual moves into a supine sleeping position creating tension that is transferred to an area within the compartment.

3. The upper body garment defined in claim 1 wherein the printed surface is a non-stretch surface.

4. The upper body garment defined in claim 1 wherein the printed surface is made of an ink solution.

5. The upper body garment defined in claim 1 wherein the printed surface is made of a gel solution.

6. The upper body garment defined in claim 1 wherein the protrusions comprises a plurality of feet.

7. The upper body garment defined in claim 6 wherein the barrier device comprises a rigid rectangle, circle, square, oval or other shaped article, with the plurality of feet.

8. The upper body garment defined in claim 1 wherein the barrier device comprises a foam insert.

9. The upper body garment defined in claim 1 further comprising a belt, strap or other device to maintain position of the barrier device.

10. The upper body garment defined in claim 1 wherein the protrusions permit airflow between portions of the barrier device and portions of the garment over which the barrier device is positioned.

11. An upper body garment having a neck opening, two sleeves or arm hole openings, a back, and a compartment located on the back of the upper body garment,
the compartment having a surface at a portion of the back that encompasses an area that includes at least the shoulder blades of an individual when worn by the individual; and
a barrier device contained within the compartment, the barrier device having plurality of feet that grip the surface when the individual initiates movement into a supine sleeping position, thereby creating tension to be applied to the surface.

12. The upper body garment defined in claim 11 wherein the surface is a non-stretch surface.

13. The upper body garment defined in claim 11 wherein the surface is a printed surface.

14. The upper body garment defined in claim 13 wherein the printed surface is made of an ink or gel.

15. The upper body garment defined in claim 11 wherein the barrier device further comprises a rigid rectangle, circle, square, oval or other shaped article, with the plurality of feet.

16. The upper body garment defined in claim 11 wherein the barrier device is a foam material.

17. The upper body garment defined in claim 11 further comprising a belt or strap to maintain position of the barrier device.

18. The upper body garment defined in claim 11 wherein the barrier device includes protrusions that permit airflow between portions of the barrier device and portions of the garment over which the barrier device is positioned.

19. The upper body garment defined in claim 11 wherein the surface comprises a fabric of material different than other portions of the garment.

20. The upper body garment defined in claim 11 wherein the surface is made of a fabric more rigid than other portions of the garment fabric.
